(19) United States
(12) Patent Application Publication
(10) Pub. No.: US 2026/0294690 A1
(43) Pub. Date: Oct. 1, 2026

(54) Wound Closure System, Kits and Method

(71) Applicant: Thomas A. Davenport, Garden City, NY (US)

(72) Inventor: Thomas A. Davenport, Garden City, NY (US)

(21) Appl. No.: 19/697,101

(22) Filed: Jun. 3, 2026

Related U.S. Application Data
(62) Division of application No. 18/665,440, filed on May 15, 2024.
(60) Provisional application No. 63/466,664, filed on May 15, 2023.

(51) Int. Cl. A61F 13/0246; A61F 13/00

(52) U.S. Cl. CPC A61F 13/0253; A61F 2013/00259

(57) ABSTRACT

Wound closure systems, kits comprising the elements of the system and the method of closing wounds using the system, said wound closure system comprising a liquid curable skin bonding adhesive component and a waterproof, natural and/or synthetic skin bonding film wound dressing or covering component.

### RELATED APPLICATIONS

The present application is a divisional application of pending U.S. patent application Ser. No. 18/665,440, filed on May 15, 2024, which claims the benefit of U.S. provisional patent application Ser. No. 63/466,664, filed on May 15, 2023.

### FIELD OF THE INVENTION

The present teaching relates to a wound closure system, kits comprising the elements of the system and the method of closing wounds using the system. Specifically, the wound closure system comprises a liquid curable skin bonding adhesive component and a waterproof, natural and/or synthetic skin bonding film wound dressing or covering component. In accordance with the present teaching, wounds are closed by applying a layer of a liquid curable skin bonding adhesive composition to the wound, incision, laceration or area of damaged skin, allowing the liquid curable skin bonding adhesive to cure, preferably to the point where it is no longer tacky, and applying a waterproof, natural and/or synthetic skin bonding film wound dressing or covering over the cured adhesive, preferably with the latter extending beyond the edges of the cured adhesive.

### BACKGROUND

The closure of wounds, especially those resulting from surgical incisions and accidental or intentional lacerations, is primarily achieved through the use of sutures, surgical staples, surgical skin tapes, and adhesive compositions. Sutures are generally recognized as providing adequate wound support for the duration of wound healing. However, suturing involves additional trauma to the wound, as the needle and suture material must be passed through the tissue at the margins of the wound. In addition, suturing can cause cosmetically unattractive wound closure marks, can be time consuming, and, depending on techniques and types of sutures used, may require removal. Such removal entails further medical attention and can involve additional pain and trauma to the patient particularly if the sutures become embedded in the wound. Surgical staples have disadvantages similar to those of sutures, including trauma to the wound, pain and cosmetic results. Further, removal of the staples can be painful and, depending on location and patient pain threshold, may require topical anesthetics. Skin closure strips, such as conventional adhesive bandages and tapes, are utilized for closure of relatively superficial skin wounds. However, the contact adhesives that are used with such strips and tapes typically retain holding power for no more than a day or two and can lose holding power quickly in the presence of moisture, for example, perspiration. Additionally, in certain individuals, the adhesive may cause skin irritation.

Though long used in battlefield situations, direct application of adhesives for wound closure is growing and becoming a preferred method due to ease of use, lack of pain and wound trauma, and more esthetically pleasing wound closure marks. Such skin bonding adhesives are primarily based upon 1,1-disubstituted ethylene monomers and polymers, such as the α-cyanoacrylates and methylidene malonates. The α-cyanoacrylates are extremely reactive, polymerizing rapidly in the presence of even minute amounts of an initiator, including moisture present in the air or on moist surfaces such as skin. The methylidene malonates are similarly extremely reactive but require the presence of a suitable activator or initiator to effect suitable cure in wound closure applications.

Commonly the use of 1,1-disubstituted ethylene adhesive compositions in medical applications, especially in wound closure applications, is as an alternative or adjunct to surgical sutures and staples in wound closure as well as for covering and protecting surface wounds such as lacerations, abrasions, burns, stomatitis, sores, and other surface wounds. When the adhesive is applied, it is usually applied in its monomeric form, and the resultant polymerization gives rise to the desired adhesive bond as well as an impermeable, sterile polymer film over the wound. For example, polymerizable 1,1-disubstituted ethylene monomers, and adhesive compositions comprising such monomers, as well as their use in medical applications, including use in wound care and/or wound closure applications, are disclosed in, e.g., U.S. Pat. Nos. 3,221,745; 3,523,907; 3,527,224; 5,328,687; 5,530,037; 5,575,997; 5,582,834; 5,624,669; 6,610,078 and 8,993,795.

Combinations of the above approaches have also been used in the art. For example, attempts have been made to combine the use of sutures or stapes and adhesive compositions as taught in, e.g., U.S. Pat. No. 5,254,132. Similarly, attempts have been made to combine the use of conventional bandages or tapes and adhesive compositions as a secondary step, as taught in, e.g., U.S. Pat. Nos. 5,259,835 and 5,445,597. However, these approaches have typically met the same issues as described above for the individual approaches, namely difficulties arising from the use of the sutures, staples and/or bandages or tapes.

While the use of the 1,1-disubstituted ethylene monomers and polymers, especially the α-cyanoacrylates and methylidene malonates, is extremely effective in wound closure, without the disadvantages of the other methods, they are not without shortcomings. Specifically, exposure to moisture, whether through perspiration or bathing, oftentimes results in the edges or perimeter of the cured film lifting from the skin surface. Even though the extent of such debonding may be minor, itching at or near these edges causes the debonding to expand deeper into the cured film. Additionally, in humans, there is often a tendency for individuals to pick at the debonded edges, again leading to further debonding if not removal of all or a significant portion of the cured adhesive film. Such extended debonding or removal may expose the underlying wound to the outside environment and/or provide a pathway for bacteria and other pathogens or environmental toxins to reach the wound itself.

Accordingly, there continues to be a need for improved materials and methods for wound approximation and closure.

### SUMMARY

According to the present teaching there is provided a wound closure system comprising a liquid curable skin bonding adhesive component and a waterproof, natural and/or synthetic skin bonding film wound dressing or covering. The liquid curable skin bonding adhesives are well known and widely available, generally comprising as its primary component a 1,1-disubstituted ethylene in monomeric, dimeric, trimeric and/or pre-polymeric form having at least one electron withdrawing substituent at the 1 position with the preferred electron withdrawing groups being selected from nitriles (including cyano), nitro, carboxylic acids, carboxylic acid esters, sulphonic acids and esters, amides, ketones and formyl; especially cyano and carboxylic acid esters, preferably cyano and carboxylic acid esters, which are capable of securely bonding and closing wounds, incisions, lacerations and the like in skin. Exemplary 1,1-disubstituted ethylenes include α-cyanoacrylates, vinylidene cyanides, alkyl homologues of vinylidene cyanide, methylidene malonates (also known as methylene malonates), acyl acrylonitriles, vinyl sulfinates, and vinyl sulfonates; most especially α-cyanoacrylates and methylidene malonates. Especially preferred 1,1-disubstituted ethylene monomers are those of Structure 2

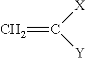

wherein X is CN or CO₂R¹ and Y is CO₂R¹ or CO₂R², wherein R¹ and R² are each independently selected from a C₁ to C₁₂, preferably C₁ to C₁₀, most preferably C₂ to C₈, straight chain or branched hydrocarbyl or heterohydrocarbyl groups: the hetero atoms being N, S or O, preferably O. Exemplary α-cyanoacrylates and methylidene malonates include, but are not limited to, alkyl α-cyanoacrylates such as 2-octyl cyanoacrylate, dodecyl cyanoacrylate, 2-ethylhexyl cyanoacrylate, n-butyl cyanoacrylate, t-butyl cyanoacrylate, ethyl cyanoacrylate, methyl cyanoacrylate, and hexyl cyanoacrylate; other α-cyanoacrylate monomers such as methoxyethyl cyanoacrylate, 2-ethoxyethyl cyanoacrylate, 3-methoxybutyl cyanoacrylate, 2-butoxyethyl cyanoacrylate, 2-isopropoxyethyl cyanoacrylate, and 1-methoxy-2-propyl cyanoacrylate; and methylidene malonates such as dimethyl methylidene malonate, diethyl methylidene malonate, di-n-butyl methylidene malonate, di-t-butyl methylidene malonate, diethyl 2-(1-methyl-ethylidene) malonate and 1-ethoxycarbonyl-1-ethoxycarbonylmethyleneoxy-carbonyl ethene, otherwise known as methylidene malonate 2.1.2 or MM2.1.2. Similarly, waterproof, natural and/or synthetic skin bonding film wound dressings or coverings are likewise well known and widely available. These are generally stretchable, flexible and/or transparent polyester, polyurethane and/or hydrocolloid films, typically having applied to one surface thereof, the bonding surface, a tacky or pressure sensitive adhesive; though other adhesives can be employed depending upon the specific application and/or the skin bonding adhesive employed therewith.

According to a second aspect of the present teaching there is provided kits for wound closure applications comprising a liquid curable skin bonding adhesive component and a waterproof, natural and/or synthetic skin bonding film wound dressing or covering. The liquid curable skin bonding adhesive is typically contained within a dispenser or applicator or in a capsule, ampoule or the like containing the liquid adhesive which capsule, ampoule or the like is to be inserted into a handheld dispenser or applicator. Especially preferred dispensers or applicators are those configured and intended for single use. The dispenser or applicator typically has associated therewith an activator or initiator for effecting or initiating the cure or polymerization of the liquid curable adhesive composition. The waterproof, natural and/or synthetic skin bonding film wound dressing or covering is present as a roll(s), sheet(s) and/or strip(s) of the dressing or covering material. Most often these are individually packaged or packaged in select numbers to maintain the sterility of the same until use. The kits may be of different configurations depending upon use and/or desire. For example, the kits may have a single or plurality of dispensers/applicators having associated therewith the liquid adhesive, a single dispenser/applicator and a plurality of ampoules, capsules or the like containing the adhesive composition and one or more rolls, tapes, sheets and/or strips of the dressing or covering material. Generally speaking, the kit would only, and preferably, have sufficient quantities of the adhesive and dressing or covering material for a single procedure so as to avoid cross-contamination from one patient to another. Similarly, a plurality of kits could be assembled to contain those quantities of the adhesive and dressing or covering material necessary for the specific procedures to be performed so as to avoid waste of unused material.

Finally, in accordance with yet another aspect of the present teaching there is provided an improved method for wound closure using a skin bonding adhesive wherein the improvement is the application of a waterproof, natural and/or synthetic skin bonding film wound dressing or covering over the cured adhesive at the site of application. Specifically, in this method, following cleaning of the wound, incision, laceration or the like, the wound edges are approximated, manually or through other means, e.g., staples, sutures, or tape, and the liquid curable adhesive is applied over the wound and adjacent areas, and allowed to cure until dry-to-the touch, i.e., no longer wet or, preferably, tacky, —as applicable for the given applicator/dispenser and method, activation or initiation of cure occurs concurrent with application or immediately preceding application of the adhesive—and then overlaying a strip or sheet of the waterproof, natural and/or synthetic skin bonding film wound dressing or covering over the cured adhesive, preferably beyond the edges of cured adhesive, and, optionally, though preferably, gently pressing on the same to ensure good contact and seal with the skin.

### DETAILED DESCRIPTION

As used herein and in the appended claims the phrase “liquid curable skin bonding adhesive” and the concept of a “skin bonding adhesive” refers to an adhesive composition that is suitable for use in closing and sealing incisions, lacerations, wounds and other skin damage where an open wound is to be closed by bringing together opposing segments of skin (i.e., approximation), including in skin grafting applications where grafted skin segments are to be bonded to each other and/or to existing skin. Though many adhesives will bond to skin, indeed, as set forth herein, the waterproof, natural and/or synthetic skin bonding film wound dressings or coverings include an adhesive, typically a tacky or pressure sensitive adhesive, which bonds them to the skin, not all, indeed, not most are suitable for use as a skin bonding adhesive. Generally, they do not create an integral cured polymer film that is able to hold the wound edges in a closed or adjacent position as required or a liquid curable skin bonding adhesive. Additionally, suitability is not limited to the physical attributes of the adhesive in creating a bond, i.e., being similar in performance to sutures and staples, but also refers to suitability from a health perspective in terms of not being toxic or damaging to the skin or the patient and/or not creating significant irritation or sensitivity in the patient or at the site of application.

As used herein, the term “flexible” is used to characterize one or more preferred characteristics or properties of the waterproof, natural and/or synthetic skin bonding film wound dressings or coverings. In particular, it is used to express that these materials are flexible, compliant, elastic, and/or memory retentive: the latter referring to the ability of the material to return to its original shape when stresses applied thereto are reduced or eliminated.

According to a first aspect of the present teaching there is provided a wound closure system comprising a liquid curable skin bonding adhesive component and a waterproof, natural and/or synthetic skin bonding film wound dressing or covering.

According to a second aspect of the present teaching there is provided kits for wound closure applications comprising a liquid curable skin bonding adhesive component and a waterproof, natural and/or synthetic skin bonding film wound dressing or covering component.

Finally, according to a third aspect of the present teaching there is provided an improved method for wound closure using a skin bonding adhesive wherein the improvement is the application of a waterproof, natural and/or synthetic skin bonding film wound dressing or covering over the cured adhesive at the site of application. Specifically, there is provided a method wherein the wound is closed using a skin bonding adhesive following which, after cure of the adhesive, a waterproof, natural and/or synthetic skin bonding film wound dressing or covering is laid over the cured adhesive, especially over the cured adhesive and a portion of the exposed skin exposed surrounding the site of application of the skin bonding adhesive.

The first critical component of the wound closure system is the liquid curable skin bonding adhesive. Such liquid curable skin bonding adhesives are well known and widely available and generally comprise as its primary component a 1,1-disubstituted ethylene in monomeric, dimeric, trimeric and/or pre-polymeric form having at least one electron withdrawing substituent at the 1 position with the preferred electron withdrawing groups being selected from nitriles (including cyano), nitro, carboxylic acids, carboxylic acid esters, sulphonic acids and esters, amides, ketones and formyl; especially cyano and carboxylic acid esters, preferably cyano and carboxylic acid esters. Suitable 1,1-disubstituted ethylene monomers typically and generally correspond to those monomers having the general Structure 1:

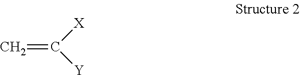

wherein R is H or a C₁ to C₆ hydrocarbyl such as methyl, ethyl, ethenyl, propyl, propenyl, isopropyl, ispropenyl, butyl, t-butyl or phenyl and X and Y are independently selected from C₁ to C₁₂, preferably C₁ to C₁₀, most preferably C₂ to C₈, hydrocarbyl or heterohydrocarbyl groups, wherein the hetero atoms are selected from N, O, and S, preferably O, and combinations thereof, provided that at least one of X and Y is a strong electron withdrawing group. Exemplary strong electron withdrawing groups include, but are not limited to, nitrile (including cyano), nitro, carboxylic acid, carboxylic acid esters, amides, ketones, sulfinates, sulfonates, and formyl with the preferred electron withdrawing groups being selected from cyano and carboxylic acid esters.

Exemplary 1,1-disubstituted ethylenes include α-cyanoacrylates, vinylidene cyanides, alkyl homologues of vinylidene cyanide, methylidene malonates (also known as methylene malonates), acyl acrylonitriles, vinyl sulfinates, and vinyl sulfonates, most especially α-cyanoacrylates and methylidene malonates. Exemplary preferred 1,1-disubstituted ethylene monomers of Structure 1 include, but are not limited to:

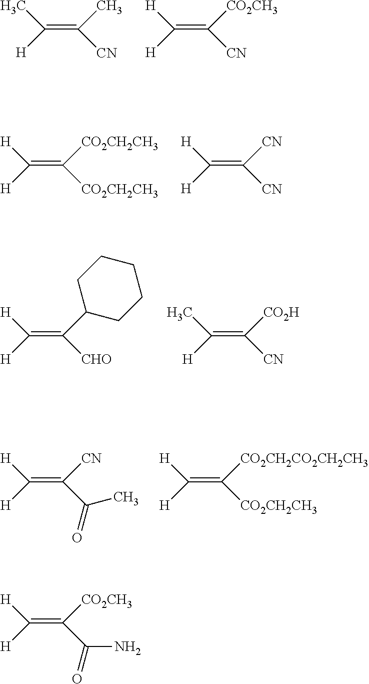

Especially preferred 1,1-disubstituted ethylene monomers are those of Structure 2

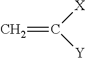

wherein X is CN or CO₂R¹ and Y is CO₂R¹ or CO₂R², wherein R¹ and R² are each independently selected from a C₁ to C₁₂, preferably C₁ to C₁₀, most preferably C₂ to C₈, straight chain or branched hydrocarbyl or heterohydrocarbyl groups: the hetero atoms being N, S or O, preferably O. Exemplary α-cyanoacrylates and methylidene malonates include, but are not limited to, alkyl α-cyanoacrylates such as 2-octyl cyanoacrylate, dodecyl cyanoacrylate, 2-ethylhexyl cyanoacrylate, n-butyl cyanoacrylate, t-butyl cyanoacrylate, ethyl cyanoacrylate, methyl cyanoacrylate, and hexyl cyanoacrylate; other α-cyanoacrylate monomers such as methoxyethyl cyanoacrylate, 2-ethoxyethyl cyanoacrylate, 3-methoxybutyl cyanoacrylate, 2-butoxyethyl cyanoacrylate, 2-isopropoxyethyl cyanoacrylate, and 1-methoxy-2-propyl cyanoacrylate; and methylidene malonates such as dimethyl methylidene malonate, diethyl methylidene malonate, di-n-butyl methylidene malonate, di-t-butyl methylidene malonate, diethyl 2-(1-methyl-ethylidene) malonate and 1-ethoxycarbonyl-1-ethoxycarbonylmethyleneoxy-carbonyl ethene, otherwise known as methylidene malonate 2.1.2 or MM2.1.2.

In addition to the polymerizable monomer, dimer, trimer and/or prepolymers, the liquid curable skin bonding adhesive composition typically includes one or more stabilizers and/or polymerization inhibitors to prevent premature polymerization in the packaging prior to use. Additionally, such compositions may include thickeners or viscosity modifies to prevent the composition from running off the patient if applied to a vertical surface; dyes to aid in visualization of the liquid as applied to the skin; softeners to make the cured adhesive more flexible; and other conventional ingredients in conventional amounts. All such additives and ingredients are well known in the art.

The liquid curable skin bonding adhesive is typically contained within a frangible chamber or ampoule within the dispenser or applicator, preferably a hand held dispenser or applicator, whereby the chamber or ampoule is fractured or otherwise compromised during use so that the liquid adhesive is able to be dispensed and applied to the wound. Alternatively, the dispenser or applicator may have a receptacle or chamber within and/or associated with its body which receives a frangible capsule, ampoule or the like containing the liquid adhesive. Typically, with those liquid skin bonding adhesives that require an activator or initiator or where the same is desirable to accelerate cure, the dispenser or applicator will also have incorporated therein the activator or initiator. Oftentimes, this is present in one or more components or passageways of the flow path, e.g., within a porous and/or channeled applicator tip; in or within the chamber that holds the ampoule or capsule whereby when the latter is compromised the liquid adhesive and the activator or initiator combine to initiate polymerization, etc., all as amply taught in the prior art. In following, as those in the art will appreciate, the dispensers or applicators may be intended or configured for single use, i.e., one discharge, or multiple use, i.e., where after the adhesive in one capsule, ampoule or the like is discharged another is inserted. Such concepts are shown in, for example, U.S. Pat. Nos. 8,794,858 and 9,487,829, among others,

As noted, liquid curable skin bonding adhesives are well known and described over and over in the technical and patent literature. Furthermore, and perhaps equally, if not more importantly, such products are widely commercially available. For example, blue dyed n-butyl-2-cyanoacrylate and blue dyed softener modified n-butyl-2-cyanoacrylate skin bonding adhesives are available from H.B. Fuller under the trademark Histoacryl. Ethicon, a Johnson & Johnson company, offers a variety of octyl cyanoacrylate skin bonding adhesives under the Dermabond trademark. Additionally, OptMed, Inc, offers or is soon to offer a methylidene malonate, specifically, a MM2.1.2, skin bonding adhesive, under the Bondease trademark. In the veterinary market there are similarly a number of commercial products including GLUture® octyl/butyl cyanoacrylate blend from Abbott Animal Health and Vetbond® n-butyl cyanoacrylate from 3M,

The skin bonding adhesive may be used alone or, depending upon the nature of the wound being closed, in combination with one or more staples or sutures which are placed to hold, i.e., approximate, the edges of the skin together while the skin bonding adhesive is applied. Alternatively, especially for large incisions, wounds, lacerations and the like, one may lay a synthetic polymer and/or natural organic polymer mesh or like permeable fabric or non-woven fabric material (collectively for convenience a “mesh”), preferably a synthetic polymer mesh, across the wound while the edges are abutted/approximated and the liquid skin bonding adhesive applied thereto so as to provide additional strength/integrity to the wound closure. Preferred meshes have a thickness of from about 0.1 to about 50 mils. Most preferably the meshes also have a preapplied pressure sensitive adhesive or tacky adhesive applied to one surface thereof so as to hold the mesh in place while the liquid curable skin bonding adhesive is applied to the same and a release polymer film or paper overlaying the preapplied tacky or pressure sensitive adhesive. The use of a mesh is especially helpful with wounds where the forces pulling at the skin, especially during subsequent movement of the patient, exceed or challenge the strength and integrity of the adhesive film formed from the liquid curable skin bonding adhesive. Optionally, the mesh has an activator or initiator for the liquid curable adhesive incorporated into or applied to the surface of the mesh whereby cure is initiated as the liquid adhesive permeates the mesh as disclosed in, e.g., US 2019/0381206 A1. An example of this product is that sold as the Dermabond® Prineo® skin bonding system by Ethicon, Inc.

The second critical component of the of the wound closure system is the waterproof, natural and/or synthetic skin bonding film wound dressing or covering. These are generally stretchable, flexible and/or transparent polymer and/or hydrocolloid films.

Preferably, these films are synthetic polymer films formed of, for example, polyolefins, e.g., polyethylene, polypropylene, ethylene propylene copolymers, and ethylene butylene copolymers; polyurethanes; polystyrenes; plasticized polyvinylchlorides; polyesters; and polyamides. Especially preferred films are those formed of polyurethanes and polyesters.

The waterproof, natural and/or synthetic skin bonding film wound dressing or covering will have applied to all or a portion of one of their surfaces an adhesive, especially a medical grade adhesive, particularly a tacky adhesive or pressure sensitive adhesive, for bonding the film to the exposed skin around the perimeter of the cured skin bonding adhesive or to both the cured skin bonding adhesive and the aforementioned perimeter area. Preferably, the adhesive substance is a medical grade adhesive, such as acrylic based pressure sensitive adhesives (PSAs), rubber based pressure sensitive adhesives, silicone pressure sensitive adhesives, mixtures thereof, or the like. Preferred pressure sensitive and/or tacky adhesives are those acrylic based polymer adhesives with sufficient tack to adhere the skin bonding film to the skin as well as to a release liner, as discussed below, and include those formed by the polymerization of at least one alkyl acrylate monomer and/or alkyl methacrylate monomer, an unsaturated carboxylic acid and, optionally, a vinyl lactam. Examples of suitable alkyl acrylate or alkyl methacrylate esters include, but are not limited to, butyl acrylate, ethyl acrylate, 2-ethylhexyl acrylate, isooctyl acrylate, isononyl acrylate, isodecyl acrylate, methyl acrylate, methylbutyl acrylate, 4-methyl-2-pentyl acrylate, sec-butyl acrylate, ethyl methacrylate, isodecyl methacrylate, methyl methacrylate, and the like, and mixtures thereof. Examples of suitable ethylenically unsaturated carboxylic acids include, but are not limited to, acrylic acid, methacrylic acid, fumaric acid, itaconic acid, and the like, and mixtures thereof. A preferred ethylenically unsaturated carboxylic acid monomer is acrylic acid. Examples of suitable vinyl lactams include, but are not limited to, N-vinyl caprolactam, 1-vinyl-2-piperidone, 1-vinyl-5-methyl-2-pyrrolidone, vinyl pyrrolidone, and the like, and mixtures thereof.

The waterproof, natural and/or synthetic skin bonding film wound dressing or covering exists in a plurality of physical formats such as rolls, tapes, sheets, strips and the like. These may be of various shapes (square, rectangular, round, oval, etc.) and dimensions, though the thickness of the films is generally from about 0.1 to about 50 mils, preferably from about 0.5 to about 20 mils, most preferably from about 1 to about 10 mils. In the case of rolls and tapes, such rolls and tapes may be, e.g., as narrow as 0.5 to about 2 inches or as wide as about 6 to about 12 inches or more, preferably as wide as about 4 to about 5 inches or so in the case of tapes, and about 24 inches or more, preferably about 36 inches or more in length. Indeed, rolls and tapes may be many feet or yards in length. Strips, whether in pre-cut form or stock materials which are to be cut to fit, are generally from about 0.5 inch, preferably from about 1 inch, to about 2 inches in width and from about 3 to about 10 or more inches in length. Similarly, sheets, whether in pre-cut form or stock materials which are to be cut to fit, are generally from about 2 inches, preferably from about 3 inches, to about 12 inches, preferably about 10 inches in width and from preferably from about 2 inches, preferably from about 3 inches, to about 12 inches, preferably about 10 inches in length. Finally, as also noted above, the sheets maybe in various shapes such as round, oval, square or rectangular: especially where the size is predetermined for a particular application or procedure.

Typically, in whatever format the films are presented, they have at least one, preferably, two additional components. The first is a release liner which may be paper, plastic or like sheet material which covers and protects the adhesive of the skin bonding film, which liner is removed prior to or as the skin bonding film is being applied to the skin. The second is a similar paper, plastic or like sheet material, a second release liner, that has its own adhesive and overlays the non-adhesive surface of the skin bonding film to protect it from damage, contamination and the like. This second release liner may be removed prior to or after application of the skin bonding film to the skin. The adhesive of this second release liner has a stronger adhesive force for the second liner material than the skin bonding film so as to completely be removed from the latter upon removal of the second release liner.

The skin bonding film wound dressings or coverings may be of one or more stock dimensions, regardless of whether they are rolls, tapes, sheets, or strips from which the desired size is cut for the given situation. In these instances, the adhesive of the wound dressing or covering will preferably cover the entirety of the bonding surface where the skin bonding wound dressing or covering is in the format of rolls or tapes. Similarly, where the skin bonding wound dressings or coverings are of stock dimensions from which a portion thereof is to be cut for the given application, it is preferred that the adhesive cover the whole of the bonding surface or, if not, a substantial portion thereof, e.g., at least 50% or more, preferably at least 60% or more, more preferably at least 70% or more so that, when cut, the entirety of that portion of the skin bonding wound dressings or coverings that extends beyond the perimeter of the cured skin bonding adhesive has the adhesive applied thereto in order to provide a good seal around the perimeter of the cured skin bonding adhesive. On the other hand, where a variety of sheets or strips of the skin bonding wound dressing or covering of set sizes are produced, each for different applications and/or lengths of a wound, laceration, incision or the like, then it is not necessary, though often desirable, for the adhesive to cover the entirety of the bonding surface. Rather, in these instances it is sufficient if the outer perimeter of said sized sheets and/or strips, i.e., that portion thereof to be in contact with the skin following application, have the skin bonding adhesive applied thereto. Here, the width of the outer perimeter should be such that at least ¼ inch, preferably at least ⅓ inch, more preferably at least ½ inch, most preferably at least ¾ inch of the perimeter of the set sized sheets and/or strips is applied to the exposed skin and not the cured skin bonding adhesive.

Although sterility of the skin bonding film wound dressings or coverings is not necessary, given concerns for contamination, particularly bacterial and/or viral contamination, the skin bonding film wound dressings or coverings, regardless of the format in which presented, are preferably sterile and maintained in a sterile environment, most especially a sterile packaging, prior to use. Such packages may contain one or more rolls, tapes, sheet or strips or the same may be individually packaged to avoid the possibility of contamination while working with one sheet.

As noted above, such skin bonding film wound dressings and coverings are well known and widely commercially available. Suitable commercial skin bonding film wound dressings and coverings, include, but are certainly not limited to, the Leukomed® T, Hypafix® and Fixomull® transparent film dressings from BSN Medical Inc., Tegaderm® transparent film dressings from 3M, Transeal® transparent film dressings from DeRoyal; DermaView® transparent film dressings from DermaRite Industries, Medline® transparent film dressings from Medline Industries, Opsite Flexifix™ transparent conformable film from Smith & Nephew, Comfeel® transparent films from Coloplast A/S, Askina Derm transparent film dressing from B Braun, among a host of others.

Although the components of the wound closure systems of the present teaching may be purchased individually, it is preferred that they be provided as a kit comprising each of the critical components, namely the liquid curable skin bonding adhesive component and the waterproof, natural and/or synthetic skin bonding film wound dressing or covering. Additionally, the kits may include one or more meshes, as described above, to be used with the skin bonding adhesive and/or other ancillary devices or materials to be employed in the wound closing procedure. With respect to the latter, the kits may also include sterile wipes to clean the wound and/or gauze to remove any exudate from the wound. The kits may be of different configurations depending upon use and/or desire. For example, the kits may have a single or plurality of dispensers/applicators having associated therewith the liquid adhesive, a single dispenser/applicator and a plurality of ampoules, capsules or the like containing the adhesive composition and one or more rolls, sheets and/or strips of the dressing or covering material. Similarly, the kits may have skin bonding film wound dressings or coverings of specific formats and/or sizes for the specific procedure for which the kit is to be employed. For example, a set of kits may be configured for use with surgical procedures having only small incisions, e.g., angioplasty, prostate removal, and other robotic surgeries. Alternatively, another set of kits may be configured for use with surgical procedures having larger incisions such as knee or hip replacement, shoulder surgery, and the like.

Generally speaking, the kit would only, and preferably, have sufficient quantities of the adhesive and dressing or covering material and, if applicable, the mesh, for a single procedure so as to avoid cross-contamination from one patient to another. Similarly, a plurality of kits could be assembled to contain those quantities of the adhesive and dressing or covering material and, if applicable, mesh material, necessary for the specific procedures to be performed so as to avoid waste of unused material. Alternatively, a more generalized kit can be produced which has multiple dispensers/applicators as well as a plurality of wound dressings for use on, e.g., an emergency vehicle, such as an ambulance, or in a clinic or small, urgent care facility or the like.

Besides the ability to customize and have in one place all the necessary elements for the wound closure procedure to be performed, kits also allow for better control on contamination, particularly bacterial or viral contamination, as the whole of the kit, once assembled, can be sterilized and packaged for use in an operating room or treatment room.

Although each of the components of the wound closure system of the present teaching is employed and applied as conventionally applied, the use of the two in combination, as presented herein, has not previously been known or employed. While skin bonding film wound dressings and coverings have been used in combination with sutures and staples, following application of the latter, to protect and isolate the wound from the environment; the films formed by the cured skin bonding adhesives employed in the present teaching already confer those benefits and properties to the wound closure. In essence, the prior art use of skin bonding adhesives, including those employed in the practice of the present teaching, provide both the closure utility of the sutures and staples as well as the protection and isolation offered by the subsequent application of the skin bonding film wound dressings and coverings over said sutures and staples. The latter are not needed to successfully provide wound closure with the skin bonding adhesives.

However, as noted above, over time, moisture and movement and manipulation of the wound area has led to some debonding of the edges of the polymer film created by the cured skin bonding adhesive. Though premature failure and sloughing off of the film is not common, itching and human nature's tendency to pick at things, exacerbates the debonding at the edges and can lead to a breach of the protective seal over the wound or the loss of coverage over part or all of the wound. In accordance with the improved wound closure method of the present teaching, this problem is eliminated as the use of the skin bonding film wound dressings and coverings prevents one from directly itching across the edges of the cure skin bonding adhesive as well as from picking or pulling at the debonded edges. Additionally, since the skin bonding film wound dressings and coverings are thin films and, preferably, transparent, the whole of the wound closure system is almost invisible: thereby avoiding unsightly and burdensome bandages. Rather, the patient is essentially able to go about their normal activities as if the wound closure system were not there.

As noted, each of the components of the current wound closure system is employed in its conventional manner, the key is that the two are applied sequentially, with the skin bonding adhesive applied first to provide wound closure and the skin bonding film wound dressings and coverings applied over the cured skin bonding adhesive. Specifically, when seeking to close a wound, incision, laceration or the like, the wound area is cleaned and any exudate removed. The edges of the wound are then approximated to assure a proper alignment and held in place while the skin bonding adhesive is dispensed and applied as a thin liquid film over the wound and its immediately surrounding area. Generally, the adhesive will extend about ¼ inch, preferably ½ inch, to 1 inch or more from each side of the wound. The edges are held until sufficient cure is achieved and cure allowed to continue until the surface of the skin bonding adhesive is no longer wet or, preferably, no longer tacky. If it is difficult or impossible to hold the wound edges in approximation or as a matter of convenience, especially for large/long wounds, one may apply one or more sutures or staples to the approximated edges to hold the same in place before applying the skin bonding adhesive. Similarly, if the situation calls for the use of a mesh to add strength and additional integrity to the wound closure, following approximation of the edges, the mesh is laid over and along the length of the wound and then the liquid skin bonding adhesive applied thereto. Again, the extent of the application of the liquid skin bonding adhesive is over the entirety of the mesh and preferably beyond its edges.

Once the skin bonding adhesive is sufficiently cured, an appropriately sized waterproof, natural and/or synthetic skin bonding film wound dressing or covering is selected or created and the release liner removed from the surface having the skin bonding adhesive thereon and the dressing or covering laid over the cured adhesive and gently pressed to the skin to ensure good contact and seal with the skin. Once in place, the second release liner, if present, is removed. In the event the stock synthetic skin bonding film wound dressing or covering is too large, the same is cut to the desired width and length to cover the cured skin bonding adhesive and its immediately surrounding area. Generally speaking, the skin bonding film wound dressing or covering will extend at least ¼ inch, preferably at least ⅓ inch, more preferably at least ½ inch, most preferably at least ¾ inch beyond the perimeter of the cured adhesive film. Although certainly allowed, the skin bonding film wound dressing or covering need not extend more than 4 inches, preferably not more than 2 inches, beyond the perimeter of the cured skin bonding adhesive.

The principles, preferred embodiments, and modes of operation of the present invention have been described in the foregoing specification. The invention which is intended to be protected herein, however, is not to be construed as limited to the particular forms disclosed, since these are to be regarded as illustrative rather than restrictive variations and charges can be made by those skilled in the art without departing from the spirit and scope of the invention. All patents and other literature cited herein are hereby incorporated herein in their entirety by this reference.

I claim:

1. A method for wound closure comprising applying a thin film of a liquid curable skin bonding adhesive over the wound and the adjacent area of the skin, allowing the liquid curable skin bonding adhesive to cure and applying a waterproof, natural and/or synthetic skin bonding film wound dressing or covering over the cured skin bonding adhesive and the area of the skin adjacent to the cured film of the skin bonding adhesive, said wound dressing or covering characterized as having edges, two surfaces and an outer perimeter.

2. The method of claim 1 wherein the liquid curable skin bonding adhesive comprises a 1,1-disubstituted ethylene in monomeric, dimeric, trimeric and/or pre-polymeric form having at least one electron withdrawing substituent at the 1 position with the preferred electron withdrawing groups being selected from nitriles (including cyano), nitro, carboxylic acids, carboxylic acid esters, sulphonic acids and esters, amides, ketones and formyl.

3. The method of claim 1 wherein the liquid curable skin bonding adhesive comprises a cyanoacrylate or methylidene malonate composition.

4. The method of claim 3 wherein the liquid curable skin bonding adhesive comprises a n-butyl cyanoacrylate, n-octyl cyanoacrylate, or an n-butyl/n-octyl cyanoacrylate blend.

5. The method of claim 1 wherein the liquid curable skin bonding adhesive comprises a n-butyl cyanoacrylate.

6. The method of claim 1 wherein the liquid curable skin bonding adhesive comprises methylidene malonate 2.1.2.

7. The method of claim 1 wherein the waterproof, natural and/or synthetic skin bonding film wound dressing or covering comprises a stretchable and flexible polymer film and/or a hydrocolloid film having a tacky adhesive or pressure sensitive adhesive on at least a portion of one surface thereof.

8. The method of claim 7 wherein the waterproof, natural and/or synthetic skin bonding film wound dressing or covering includes a release polymer liner or paper overlying the adhesive and the method further comprises removing the liner or paper prior to applying the film wound dressing or covering.

9. The method of claim 1 wherein the waterproof, natural and/or synthetic skin bonding film wound dressing or covering comprises a stretchable and flexible polyethylene film, polypropylene film, polyurethane film, polyester film or hydrocolloid film of from about 0.1 to about 50 mils in thickness and the tacky adhesive or pressure sensitive adhesive is present along the outer perimeter of the surface of the wound dressing or covering.

10. The method of claim 1 further comprising applying a polymer mesh or permeable woven or non-woven fabric material to the surface of the wound prior to applying the liquid curable skin bonding adhesive.

11. The method of claim 10 wherein polymer mesh or permeable woven or non-woven fabric material is applied over the surface of the wound, with or without its edges overlaying the surface of the skin adjacent to the wound.

12. The method of claim 10 wherein the polymer mesh or permeable woven or non-woven fabric material has a pre-applied tacky or pressure sensitive adhesive on at least a portion of one surface thereof for securing the polymer mesh or permeable woven or non-woven fabric material to the skin adjacent the wound.

13. The method of claim 1 wherein the edges of the wound are approximated prior to applying the liquid curable skin bonding adhesive.

14. The method of claim 13 wherein the edges of the wound are approximated by the use of one or more sutures and/or staples and/or a polymer mesh or permeable woven or non-woven fabric material, wherein polymer mesh or permeable woven or non-woven fabric material has a tacky or pressure sensitive adhesive on at least a portion of one surface thereof for securing the polymer mesh or permeable woven or non-woven fabric material to the skin adjacent the wound.

15. The method of claim 13 wherein the edges of the wound are approximated by the use of one or more sutures and/or staples.

16. The method of claim 15 wherein following approximation with the sutures or staples, a polymer mesh or permeable woven or non-woven fabric material is applied over the wound prior to applying the liquid curable adhesive.

17. The method of claim 13 wherein the edges of the wound are approximated by the use of a polymer mesh or permeable woven or non-woven fabric material having a tacky or pressure sensitive adhesive on at least a portion of one surface thereof for securing the polymer mesh or permeable woven or non-woven fabric material to the skin adjacent the wound.

18. The method of claim 17 wherein the approximation is performed with a polymer mesh.

19. The method of claim 1 wherein the liquid curable skin bonding adhesive comprises a cyanoacrylate or methylidene malonate composition and the waterproof, natural and/or synthetic skin bonding film wound dressing or covering comprises a stretchable and flexible polyethylene film, polypropylene film, polyurethane film, polyester film or hydrocolloid film of from about 0.1 to about 50 mils in thickness and having a tacky adhesive or pressure sensitive adhesive on at least a portion of the surface thereof to be applied to the skin.

20. The method of claim 19 wherein the edges of the wound are approximated by the use of one or more sutures and/or staples and/or a polymer mesh or permeable woven or non-woven fabric material, wherein polymer mesh or permeable woven or non-woven fabric material has a tacky or pressure sensitive adhesive on at least a portion of one surface thereof for securing the polymer mesh or permeable woven or non-woven fabric material to the skin adjacent the wound.
